(19) United States
(12) Patent Application Publication
(10) Pub. No.: US 2004/0260131 A1
(43) Pub. Date: Dec. 23, 2004

(54) Selective hydrocarbon hydrogenation catalyst and process

(75) Inventors: Joseph J. Bergmeister, Kingwood, TX (US); Gary A. Delzer, Galveston, TX (US); Tin-Tack P. Cheung, Kingwood, TX (US)

Correspondence Address: CHEVRON PHILLIPS CHEMICAL COMPANY LP, LAW DEPARTMENT - IP, P.O BOX 4910, THE WOODLANDS, TX, 77387-4910, US

(73) Assignee: Chevron Phillips Chemical Company ("CPCHEM")

(21) Appl. No.: 10/600,609

(22) Filed: Jun. 23, 2003

(51) Int. Cl. C07C 5/03

(52) U.S. Cl. 585/259

(57) ABSTRACT

This invention relates to acetylene removal catalysts and their use in the hydrogenating of highly unsaturated hydrocarbons to less unsaturated hydrocarbons in an olefin rich hydrocarbon stream in the presence of hydrogen and a catalyst composition under conditions effective to convert said highly unsaturated hydrocarbon to a less unsaturated hydrocarbon. Said catalyst composition comprises palladium, silver, potassium, and an inorganic support material, wherein the catalyst composition contains less than about 0.3 weight % potassium. In the presence of sulfur-containing impurities, the catalysts of the present invention yield a much smaller increase in T1 (cleanup temperature) and higher ethylene selectivity is achieved.

### FIELD OF THE INVENTION

This invention relates to acetylene removal catalysts and their improved process for hydrogenation of hydrocarbons. In another aspect, this invention relates to processes for hydrogenation of hydrocarbons generally and particularly selectively hydrogenating alkynes and/or diolefins to their corresponding monoolefins employing palladium/silver/alumina catalysts, impregnated with potassium compound. This invention also relates to improved processes for hydrogenation of hydrocarbons employing potassium fluoride impregnated palladium/silver/alumina catalysts in the presence of sulfur-containing impurities in a depropanizer feed. In the presence of sulfur-containing impurities, the catalyst of the present invention is more active and achieves higher ethylene selectivity.

### BACKGROUND OF THE INVENTION

The selective hydrogenation of alkynes, which generally are present in small amounts in alkene-containing streams (e.g., acetylene contained in ethylene streams from thermal ethane crackers), is commercially carried out in the presence of supported palladium catalysts. In the case of the selective hydrogenation of acetylene to ethylene, preferably an alumina-supported palladium/silver catalyst is used in accordance with the disclosure in U.S. Pat. No. 4,404,124 and its division, U.S. Pat. No. 4,484,015. The operating temperature for this hydrogenation process is selected such that essentially all acetylene is hydrogenated to ethylene (and thus removed from the feed stream) while only an insignificant amount of ethylene is hydrogenated to ethane to minimize ethylene losses and to avoid a “runaway” reaction which is difficult to control, as has been pointed out in the above-identified patents.

It is also generally known to those skilled in the art that sulfur-containing impurities, such as H₂S, carbonyl sulfide (COS), mercaptans (RSH), organic sulfides (R—S—R), organic disulfides (R—S—S—R), organic polysulfides (R—Sₙ—R, where n>2), and the like, which can be present in an alkyne-containing feed or product stream, can poison and deactivate a palladium-containing catalyst. Since many plants have various sulfur impurities continuously present or at least present as intermittent spikes, it would be advantageous to be able to run both in the presence of and absence of such various sulfur impurities. Sulfur impurities are usually found in depropanizer and raw gas hydrogenation processes (but can occur in any hydrogenation process) as a result of plant and operational limitations. The feed stream being hydrogenated can contain either low levels and/or transient spikes of a sulfur impurity. Thus, the development of a catalyst composition for use in a front-end depropanizer ARU ethylene plant for the hydrogenation of highly unsaturated hydrocarbons such as diolefins (alkadienes) or alkynes to less unsaturated hydrocarbons such as monoolefins (alkenes), both in the presence of and in the absence of a sulfur impurity, would be a significant contribution to the art and to the economy.

Other aspects and features of the invention will become apparent from review of the detailed description and the claims.

### DETAILED DESCRIPTION OF THE INVENTION

The catalyst which is employed in the selective hydrogenation process of this invention is a supported palladium catalyst composition which comprises a silver component and lower levels of a potassium component and optionally a fluorine component. This catalyst composition can be fresh or it can be a previously used and thereafter oxidatively regenerated. This catalyst can contain any suitable inorganic solid support material. Preferably, the inorganic support material is selected from the group consisting of alumina, titania, zirconia, and mixtures thereof. The presently more preferred support material is alumina, most preferably alpha-alumina. This catalyst generally contains palladium, a silver component, a fluorine component, and a potassium component. Wherein the weight % palladium is selected from one of the following ranges 0.01-1, 0.01-0.6, 0.01-0.2, 0.01-0.1, etc. Wherein the weight % of silver is selected from one of the following ranges 0.005-10, 0.01-10, 0.005-2, 0.01-2, etc. Wherein the weight % fluorine is selected from one of the following ranges 0.01-1.5, 0.05-0.4, etc. Wherein the weight % of potassium is selected from one of the following ranges, less than 0.3, less than 0.2, less than 0.1, etc. weight % potassium. Particles of this catalyst generally have a size of 1-10 mm (preferably 2-6 mm) and can have any suitable shape. Suitable shapes can be selected from spherical, cylindrical, extrudates, multilobe extrudates, etc. Generally, the surface area of this catalyst (determined by the BET method employing N₂) is 1-100 m²/g.

The above-described catalyst which is employed in the hydrogenation process of this invention can be prepared by any suitable, effective method. The potassium fluoride can be incorporated between the palladium and the silver impregnation steps after the palladium and silver impregnation steps or together with either the palladium or silver. The presently preferred catalyst preparation comprises the impregnation of a Pd/Ag/Al₂O₃ catalyst material with an aqueous solution of potassium fluoride, followed by drying and calcining. The drying and calcining step occurs in an atmosphere of any inert gas containing from 0.1 to 100 volume % oxygen, at a temperature selected from one of the following ranges 300-800° C., 350-600° C., etc, generally for 0.1-20 hours. It is possible, to apply a “wet reducing” step (i.e., treatment with dissolved reducing agents such as hydrazine, alkali metal borohydrides, aldehydes such as formaldehyde, carboxylic acids such as forming acid or ascorbic acid, reducing sugars such as dextrose, and the like).

The thus-prepared catalyst composition which has been dried (and preferably also calcined, as described above) can then be employed in the process of this invention for hydrogenating at least one alkyne, preferably acetylene, to at least one corresponding alkene in both the presence and absence of at least one sulfur compound. Optionally, the catalyst is first contacted, prior to the alkyne hydrogenation, with hydrogen gas optionally diluted with 0-95 volume % of any gas substantially free of unsaturated hydrocarbons, generally at a temperature in the range of 20° C. to 100° C., for a time period of 1 to 20 hours. During this contacting with hydrogen before the selective alkyne hydrogenation commences, palladium and silver compounds which may be present in the catalyst composition after the drying step and the optional calcining step (described above) are substantially reduced to palladium and silver metal. When this optional reducing step is not carried out, the hydrogen gas present in the reaction mixture accomplishes this reduction of oxides of palladium and silver during the initial phase of the alkyne hydrogenation reaction of this invention.

The selective hydrogenation process of this invention is carried out by contacting highly unsaturated hydrocarbons, hydrogen gas, optionally in the presence of one or more sulfur-containing impurities with the inventive catalyst composition. These components are reacted under conditions effective in converting the highly unsaturated hydrocarbons to less unsaturated hydrocarbons in a front-end depropanizer ARU.

The term “highly unsaturated hydrocarbon” refers to a hydrocarbon having one (or more) triple bond(s) or two or more double bonds between carbon atoms in the molecule. Examples of highly unsaturated hydrocarbons include, but are not limited to, aromatic compounds such as benzene and naphthalene; alkynes such as acetylene, propyne (also referred to as methylacetylene), and butynes; diolefins such as propadiene, butadienes, pentadienes (including isoprene), hexadienes, octadienes, and decadienes; and the like and mixtures thereof. The term “less unsaturated hydrocarbon” refers to a hydrocarbon in which the one (or more) carbon-to-carbon triple bond(s) in a highly unsaturated hydrocarbon is (are) hydrogenated to a carbon-to-carbon double bond(s), or a hydrocarbon in which the number of carbon-to-carbon double bonds is one less, or at least one less, than that in a highly unsaturated hydrocarbon, or a hydrocarbon having at least one carbon-to-carbon double bond. Examples of less unsaturated hydrocarbons include, but are not limited to, monoolefins such as ethylene, propylene, butenes, pentenes, hexenes, octenes, decenes, and the like and mixtures thereof.

During the selective hydrogenation process of the present invention, a hydrocarbon feed containing at least one highly unsaturated hydrocarbon and hydrogen, optionally in the presence of sulfur-containing impurities, are fed to an Acetylene Hydrogenation Unit, where the catalyst composition of the present invention resides.

The highly unsaturated hydrocarbon includes diolefins, alkynes, and mixtures of two or more thereof.

Alkynes include acetylene, propyne, 1-butyne, 2-butyne, 1-pentyne, 2-pentyne, 3-methyl-1-butyne, 1-hexyne, 1-heptyne, 1-octyne, 1-nonyne, 1-decyne, and mixtures thereof. Particularly preferred is acetylene. These alkynes are primarily hydrogenated to the corresponding alkenes, i.e., acetylene is primarily hydrogenated to ethylene, propyne is primarily hydrogenated to propylene, and the butynes are primarily hydrogenated to the corresponding butenes (1-butene, 2-butene).

Diolefins include propadiene, 1,2-butadiene, 1,3-butadiene, isoprene, 1,2-pentadiene, 1,3-pentadiene, 1,4-pentadiene, 1,2-hexadiene, 1,3-hexadiene, 1,4-hexadiene, 1,5-hexadiene, 2-methyl-1,2-pentadiene, 2,3-dimethyl-1,3-butadiene, heptadienes, methylhexadienes, octadienes, methylheptadienes, dimethylhexadienes, ethylhexadienes, trimethylpentadienes, methyloctadienes, dimethylheptadienes, ethyloctadienes, trimethylhexadienes, nonadienes, decadienes, undecadienes, dodecadienes, cyclopentadienes, cyclohexadienes, methylcyclopentadienes, cycloheptadienes, methylcyclohexadienes, dimethylcyclopentadienes, ethylcyclopentadienes, dicyclopentadiene, and mixtures thereof. More preferably, the diolefin is propadiene, 1,3-butadiene, 1,3-pentadiene, 1,4-pentadiene, isoprene, 1,3-cyclopentadiene, dicyclopentadiene, and mixtures thereof. Particularly preferred is propadiene.

The temperature necessary for the selective hydrogenation of alkyne(s) to alkene(s) depends largely upon the activity and selectivity of the catalysts, the amounts of sulfur impurities in the feed, and can be any suitable temperature to achieve the desired extent of alkyne removal. Generally, a reaction temperature in the range of about 30° C. to about 200° C. is employed. Any suitable reaction pressure can be employed. Generally, the total pressure is in the range of 100 to 1,000 pounds per square inch gauge (psig). The gas hourly space velocity (GHSV) of the hydrocarbon feed gas can also vary over a wide range. Typically, the gas hourly space velocity will be in the range of about 1,000 to 20,000.

Regeneration of the catalyst composition can be accomplished by heating the catalyst composition in an atmosphere of any inert gas containing from 0.1 to 100 volume % oxygen at a temperature which preferably does not exceed 700° C. so as to burn off any sulfur compounds, organic matter and/or char that may have accumulated on the catalyst composition. Optionally, the oxidatively regenerated composition is reduced with hydrogen diluted with 0 to 95 volume % of any gas substantially free of unsaturated hydrocarbons before its redeployment in the selective alkyne hydrogenation of this invention.

The following examples are presented to further illustrate this invention and are not to be construed as limiting its scope.

### EXAMPLE I

This example illustrates the preparation of various palladium-containing catalyst compositions to be used in a hydrogenation process.

Catalyst A (Control) was prepared in accordance with U.S. Pat. No. 5,489,565 and contained 0.014 weight % Pd, 0.044 weight % Ag, 0.3 weight % K, and 0.15 weight % F on aluminum oxide support.

Catalyst B (Control) was prepared in accordance with U.S. Pat. No. 5,587,348 and contained 0.013 weight % Pd, 0.044 weight % Ag, 0.3 weight % K, and 0.3 weight % F on aluminum oxide support.

Catalyst C (Invention) was prepared in accordance with U.S. Pat. No. 5,489,565 and contained 0.02 weight % Pd, 0.04 weight % Ag, 0.1 weight % K, and 0.05 weight % F on aluminum oxide support.

### EXAMPLE II

This example illustrates the performance of the catalysts described hereinabove in Example I in a hydrogenation process in the absence and the presence of sulfur.

About 23 grams (i.e., about 20 cc) of each of the above described catalysts were placed in a stainless steel reactor tube having a 0.62 inch inner diameter and a length of about 18 inches. The catalyst (resided in the middle of the reactor; both ends of the reactor were packed with 6 mL of 3 mm glass beads) was reduced at about 100° F. for about 1 hour under hydrogen gas flowing at 200 mL/min at 200 pounds per square inch gauge (psig). Thereafter, a hydrocarbon-containing fluid, typical of a feed from the top of a depropanizer fractionation tower in an ethylene plant, containing approximately (all by weight unless otherwise noted) hydrogen, 2.1%; methane, 22%; ethylene, 54%; propylene, 21%; acetylene, 4300 ppm; propadiene, 4300 ppm; propyne, 4300 ppm; and carbon monoxide, 300 ppm (by volume) was continuously introduced into the reactor at a flow rate of 900 mL per minute at 200 psig. The reactor temperature was increased until the hydrogenation ran away, i.e., the uncontrollable hydrogenation of ethylene was allowed to occur. During the runaway, the heat of hydrogenation built up such that the reactor temperature exceeded about 250° F. The reactor was then allowed to cool to room temperature before data collection was started.

Feed (900 mL/min @ 200 psig) was passed over the catalyst continuously while holding the temperature constant before sampling the exit stream by gas chromatography. The catalyst temperature was determined by inserting a thermocouple into the thermowell and varying its position until the highest temperature was observed, the furnace was then raised a few degrees, and the testing cycle was repeated until 3 weight % of ethane was produced.

The cleanup temperature, T1, is defined as the temperature at which the acetylene concentration drops below 20 ppm. The T2, runaway temperature, is defined as the temperature at which 3 wt % of ethane is produced. At this temperature the uncontrolled hydrogenation of ethylene to ethane begins. And delta T is the difference between T2 and T1. This value can be viewed as a measure of selectivity or even a window of operability.

Each catalyst was exposed to the high carbonyl sulfide (COS) concentration at different temperatures. This was determined by predicting what the T1_{cos} would be. By exposing the catalyst to the high concentration of COS at a temperature of 10° F. less than the predicted T1_{cos}, the amount of time it took for the reaction to reach a steady state was minimized.

The T1_{cos} was determined as follows. First 12 ppm COS was added to the feed and the flow rate was lowered to 90 mL/min. A 300 mL (STP) portion of 5000 ppm COS in nitrogen was then introduced into the feed stream. After 5 minutes the flow rate was returned to 900 mL/min. The COS was introduced with a low flow rate to ensure there was sufficient contact time between the COS and the catalyst.

After over exposing the catalyst to COS, the reactor temperature was held constant until the acetylene concentration in the exit stream reached a steady state. At this point the reactor temperature was either lowered or raised to determine T1_{cos}. The entire run was conducted in a continuous mode, sulfur containing hydrocarbon feed always in contact with the catalyst. The reactor effluent, i.e., the product stream, was analyzed by gas chromatography. The results are shown in Table I. In addition, in Table I “hydrocarbon selectivities at T1” refers to the percent of acetylene that was transformed to its corresponding hydrocarbon at T1. Selectivities were determined on a mole basis.

TABLE 1

| | | | F:K | | | Delta | Selectivity to | | | | |
|---|---|---|---|---|---|---|---|---|---|---|---|
| | | COS | molar | T1 | T2 | T | C2 | C4's | C5's | heavies | C2= |
| Run | Catalyst | (ppmv) | ratio | (° F.) | (° F.) | (° F.) | (%) | (%) | (%) | (%) | (%) |
| 101 | A | 0 | 1 | 151 | 225 | 74 | 14.5 | 12.2 | 4.3 | 3.3 | 65.8 |
| 102 | A | 12 | 1 | 248 | * | * | 110.7 | 2.8 | 1.6 | 0 | −15.1 |
| 103 | B | 0 | 2 | 149 | 218 | 69 | 16.1 | 10.6 | 6.1 | 3.9 | 63.3 |
| 104 | B | 12 | 2 | 203 | * | * | 78.4 | 3.9 | 2.1 | 0 | 15.7 |
| 105 | C | 0 | 1 | 132 | 186 | 54 | 16.6 | 12.5 | 7.6 | 5.5 | 57.8 |
| 106 | C | 12 | 1 | 177 | * | * | 75.5 | 4.6 | 0.8 | 0 | 19.1 |

* = not determined
C2 - ethane
C4's - any hydrocarbon with 4 carbons
C5's - any hydrocarbon with 5 carbons
heavies - any hydrocarbon with 6 or more carbons
C2= - ethylene

Comparing run 101 to 103 there is little difference in the performance of catalyst A and B in the absence of sulfur. However runs 102 and 104 demonstrate that the additional fluorine on the catalyst improves the ethylene selectivity by 30%.

Comparing run 105 to 101 and 103, the only difference between the two runs in the absences of sulfur's. T1 for run 105 is lower. When sulfur is present, catalyst C (run 106) has an ethylene selectivity 39% better than catalyst A (run 102) and similar to catalyst B (run 104).

Thus these examples show that decreasing the total potassium concentration eliminates the need for additional fluorine on the catalyst.

While the foregoing discussion is intended to provide a detailed illustration of certain embodiments of the invention, it will be appreciated that additional embodiments are also possible under the claims provided herein. It will also be appreciated that numerical values and ranges are presented in approximate form such that small or inconsequential deviations from such values are intended to be within the spirit and scope of the values and ranges presented.

What is claimed is:

1. A process for selectively hydrogenating a highly unsaturated hydrocarbon to a less unsaturated hydrocarbon in an olefin rich hydrocarbon stream comprising introducing into a reactor, from a fractionation tower, a hydrocarbon fluid stream comprising a highly unsaturated hydrocarbon in the presence of hydrogen and a catalyst composition under conditions effective to convert said highly unsaturated hydrocarbon to a less unsaturated hydrocarbon;
said catalyst composition comprising palladium, silver, potassium, and an inorganic support material, wherein the catalyst composition contains less than about 0.3 weight % potassium.

2. The process according to claim 1, wherein the potassium component is derived from potassium fluoride.

3. The process according to claim 2, wherein a molar ratio of potassium to fluoride is less than 2:1.

4. The process according to claim 2, wherein a molar ratio of potassium to fluoride is less than 2:1.

5. The process according to claim 1, wherein said catalyst composition contains less than 0.2 weight % potassium.

6. The process according to claim 4, wherein said catalyst composition contains 0.1 weight % potassium.

7. The process according to claim 1, wherein said silver is selected from the group consisting of silver oxide and silver metal.

8. The process according to claim 1, wherein said inorganic support material is selected from the group consisting of alumina, silica, titania, zirconia, aluminosilicates, zinc aluminate, zinc titanate, and mixtures thereof.

9. The process according to claim 8, wherein said inorganic support material is alumina.

10. The process according to claim 1, wherein the palladium content is 0.01-1 weight %, the silver content is 0.01-10 weight %, and the fluorine content is 0.01-1.5 weight %.

11. The process according to claim 10, wherein the palladium content is 0.01-0.2 weight %, the silver content is 0.02-2 weight %, and the fluorine content is 0.05-0.4 weight %.

12. The process according to claim 1, wherein said highly unsaturated hydrocarbon is selected from the group consisting of diolefins, alkynes, and mixtures thereof.

13. The process according to claim 12, wherein said diolefin is selected from the group consisting of propadiene, 1,2-butadiene, 1,3-butadiene, isoprene, 1,2-pentadiene, 1,3-pentadiene, 1,4-pentadiene, 1,2-hexadiene, 1,3-hexadiene, 1,4-hexadiene, 1,5-hexadiene, 2-methyl-1,2-pentadiene, 2,3-dimethyl-1,3-butadiene, heptadienes, methylhexadienes, octadienes, methylheptadienes, dimethylhexadienes, ethylhexadienes, trimethylpentadienes, methyloctadienes, dimethylheptadienes, ethyloctadienes, trimethylhexadienes, nonadienes, decadienes, undecadienes, dodecadienes, cyclopentadienes, cyclohexadienes, methylcyclopentadienes, cycloheptadienes, methylcyclohexadienes, dimethylcyclopentadienes, ethylcyclopentadienes, dicyclopentadiene, and mixtures thereof.

14. The process according to claim 13, wherein said diolefin is selected from the group consisting of propadiene, 1,3-butadiene, 1,3-pentadiene, 1,4-pentadiene, isoprene, 1,3-cyclopentadiene, dicyclopentadiene, and mixtures thereof.

15. The process according to claim 14, wherein said diolefin is propadiene.

16. The process according to claim 12, wherein said alkyne is selected from the group consisting of acetylene, propyne, 1-butyne, 2-butyne, 1-pentyne, 2-pentyne, 3-methyl-1-butyne, 1-hexyne, 1-heptyne, 1-octyne, 1-nonyne, 1-decyne, and mixtures thereof.

17. The process according to claim 16, wherein said alkyne is selected from the group consisting of acetylene, propyne, and mixtures thereof.

18. The process according to claim 1, wherein said process further comprises the presence of a sulfur impurity.

19. The process according to claim 18, wherein said sulfur impurity is a sulfur compound selected from the group consisting of hydrogen sulfide, carbonyl sulfide (COS), carbon disulfide (CS₂), mercaptans (RSH), organic sulfides (R—S—R), organic disulfides (R—S—S—R), organic polysulfides (R—Sₙ—R, n where >2), thiophene, substituted thiophenes, organic trisulfides, organic tetrasulfides, and mixtures thereof, wherein R represents an alkyl or cycloalkyl or aryl group containing 1 carbon atom to 10 carbon atoms.

20. A process comprising introducing into a reactor, from a depropanizer fractionation tower, a fluid stream comprising an alkyne and optionally a diolefin, in the presence of hydrogen and a catalyst composition, under conditions effective to convert said diolefin and alkyne to their corresponding monoolefins;
said catalyst composition comprises palladium, a silver component, a potassium compound, and an inorganic support material; wherein said catalyst composition contains less than 0.3 weight % potassium;
said diolefin is selected from the group consisting of propadiene, 1,3-butadiene, 1,3-pentadiene, 1,4-pentadiene, isoprene, 1,3-cyclopentadiene, dicyclopentadiene, and mixtures thereof;
said alkyne is selected from the group consisting of acetylene, propyne, 1-butyne, 2-butyne, 1-pentyne, 2-pentyne, 3-methyl-1-butyne, 1-hexyne, 1-heptyne, 1-octyne, 1-nonyne, 1-decyne, and mixtures thereof;
said inorganic support material is selected from the group consisting of alumina, silica, titania, zirconia, aluminosilicates, zinc aluminate, zinc titanate, and mixtures thereof.

21. The process according to claim 20, wherein a molar ratio of potassium to fluoride is less than 2:1.

22. The process according to claim 21, wherein the molar ratio of potassium to fluoride is less than 2:1.

23. The process according to claim 20, wherein said catalyst composition contains less than 0.2 weight % potassium.

24. The process according to claim 23, wherein said catalyst composition contains 0.1 weight % potassium.

25. The process according to claim 20, wherein said silver component is selected from the group consisting of silver oxide and silver metal.

26. The process according to claim 20, wherein the palladium content is 0.01-1 weight %, the silver component is 0.01-10 weight %, and the fluorine content is 0.01-1.5 weight %; and
said highly unsaturated hydrocarbon is selected from the group consisting of acetylene, propadiene, 1,3-butadiene, 1,3-pentadiene, 1,4-pentadiene, isoprene, 1,3-cyclopentadiene, dicyclopentadiene, and mixtures thereof.

27. The process according to claim 26, wherein the palladium content is 0.01-0.2 weight %, the silver component is 0.01-2 weight %, and the fluorine content is 0.05-0.4 weight %.

28. The process according to claim 20, wherein said process further comprises the presence of a sulfur impurity.

29. The process according to claim 28, wherein said sulfur impurity is a sulfur compound selected from the group consisting of hydrogen sulfide, carbonyl sulfide (COS), carbon disulfide (CS₂), mercaptans (RSH), organic sulfides (R—S—R), organic disulfides (R—S—S—R), organic polysulfides (R—Sₙ—R, n where >2), thiophene, substituted thiophenes, organic trisulfides, organic tetrasulfides, and mixtures thereof, wherein R represents an alkyl or cycloalkyl or aryl group containing 1 carbon atom to 10 carbon atoms.

30. A selective hydrogenation process comprising introducing into a reactor, from a depropanizer fractionation tower, a fluid stream comprising a diolefin and acetylene, optionally in the presence of a sulfur impurity, with a catalyst composition under conditions effective to convert said diolefin and acetylene to their corresponding monoolefins
said catalyst composition comprises a palladium-containing material selected from the group consisting of palladium metal, palladium oxides, and mixtures thereof, a silver component, an alkali metal fluoride, and an inorganic support material;
said alkali metal fluoride is potassium fluoride and said inorganic support material is selected from the group consisting of alumina, silica, titania, zirconia, aluminosilicates, zinc aluminate, zinc titanate, and mixtures thereof;
said catalyst composition contains 0.01 to 1 weight % palladium, 0.005 to 2 weight % of a silver component, 0.05-0.4 weight % fluorine; and less than 0.3 weight % potassium;
said process is carried out at a temperature in the range of 30 to 200° C. and under a pressure in the range of 15 to 2000 pounds per square inch gauge (psig).

31. The process according to claim 30, wherein a molar ratio of potassium to fluoride is less than 2:1.

32. The process according to claim 31, wherein the molar ratio of potassium to fluoride is 1:1.

33. The process according to claim 30, wherein said catalyst composition contains less than 0.2 weight % potassium.

34. The process according to claim 33, wherein said catalyst composition contains 0.1 weight % potassium.

35. The process according to claim 30, wherein said inorganic support material is alumina.

36. The process according to claim 30, wherein said sulfur impurity is a sulfur compound selected from the group consisting of hydrogen sulfide, carbonyl sulfide (COS), carbon disulfide (CS₂), mercaptans (RSH), organic sulfides (R—S—R), organic disulfides (R—S—S—R), organic polysulfides (R—Sₙ—R, n where >2), thiophene, substituted thiophenes, organic trisulfides, organic tetrasulfides, and mixtures thereof, wherein R represents an alkyl or cycloalkyl or aryl group containing 1 carbon atom to 10 carbon atoms.
